(19) United States
(12) Patent Application Publication
(10) Pub. No.: US 2010/0036378 A1
(43) Pub. Date: Feb. 11, 2010

(54) THERMAL IMAGING FEEDBACK FOR OPTIMIZING RADIO FREQUENCY ABLATION THERAPY

(75) Inventors: David L.M. Savery, Calas (FR); Christopher S. Hall, Hopewell Junction, NY (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/446,797

(22) PCT Filed: Oct. 19, 2007
(86) PCT No.: PCT/IB2007/054269

§ 371 Date: Apr. 23, 2009

Related U.S. Application Data
(60) Provisional application No. 60/862,621, filed on Oct. 24, 2006.

(51) Int. Cl. A61B 18/14; A61B 5/055; A61B 8/12

(52) U.S. Cl. 606/42; 600/411; 600/439

(57) ABSTRACT

The invention relates to methods and systems for monitoring and regulating radiofrequency ablation therapy in order to maximize effectiveness of treatment. The invention uses an imaging scanner to provide feedback regarding location and extent of a treated volume. The feedback is used as input data for control of intensity, duration, and/or placement of radiofrequency treatment. Control of treatment parameters is automatic and/or modulated by an operator.

The technical field of the invention herein is methods and systems for monitoring and regulating radiofrequency ablation therapy.

New curative interventional therapies have recently emerged for treatment of cancer. One such therapy, radiofrequency (RF) ablation, has produced promising results in the treatment and management of unresectable tumors, such as in liver tissue. When tumor size is limited (e.g. diameter <3 cm), the tumor recurrence rate following RF ablation is comparable to that of tumors treated surgically. However, for bigger tumors, the recurrence rate following RF ablation is elevated, primarily as a result of incomplete necrosis of the tumor. Success of RF procedures relies on accurate deposition of a thermal dose into a cancerous lesion, while sparing healthy tissue in order to minimize side effects. In order to maximize therapeutic success for larger tumors, it is important that treatment both be thorough and have adequate control of RF power.

Currently, most commercial RF systems have a control feedback mechanism that uses input parameters given by electrical impedance or temperature measurements, from one or more thermocouples embedded near the tip of the RF probe. A primary goal of the feedback mechanism is to prevent overheating of tissue in specific areas where the thermocouples are located. However, it has not been possible to make decisions based on information of the actual state of the tissue or of the spatial extent of the necrosed volume using these systems.

Recent studies have also shown that thermocouples are not ideal devices for use with thermal therapies such as RF ablation, as thermocouples are invasive and measure temperature only at predetermined sensor locations (which might not correspond to actual tissue locations of interest). Furthermore, thermocouples are not well suited for high-intensity ultrasound surgery procedures (another type of thermal therapy) due to the ease with which they are damaged and because they often act as unwanted scatterers of ultrasound waves (R. Seip, E S. Ebbini, IEEE Transactions on Biomedical Engineering. Vol. 42, no. 8, pp 828-839, 1995).

Accordingly, an exemplary embodiment of the invention herein is a method for monitoring and regulating radiofrequency (RF) ablation therapy, having the following steps: providing an image of a target volume and surrounding tissue using an imaging scanner, and inserting an RF probe into the target volume; generating an RF current to heat the target volume near at least one tip of the RF probe, using imaging data as feedback; and altering at least one of the following parameters: RF power, exposure time, and/or position of the RF probe, in response to an imaging feedback signal transmitted to an RF power generator and/or displayed to an operator.

In a related embodiment of the above method, the scanner comprises an ultrasound scanner. In an alternative related embodiment, the scanner comprises a magnetic resonance scanner or the scanner comprises both an ultrasound scanner and a magnetic resonance scanner. In another related embodiment, the image is an ultrasound real-time image and/or a magnetic resonance image. In yet another related embodiment, the image guides inserting or adjusting placement of the RF probe in the target volume. A related embodiment includes a boundary of the target volume, which is determined by an automatic method or a manual method.

In a related embodiment of the above method, altering the parameter provides computing a temperature elevation in the target volume. In another related embodiment, a temperature elevation is derived from the image. In yet another related embodiment, the temperature elevation is measured through temperature-dependent changes in imaging properties of tissue.

A related embodiment of the above method provides altering the parameter by computing an estimated accumulated thermal dose. In another related embodiment, the estimated accumulated thermal dose is derived from the image.

Another related embodiment of the above method provides altering the parameter by estimating a location and/or a volume of a thermally affected region, to calculate an estimated coagulated volume (ECV). In another related embodiment, the ECV is derived from the image.

Yet another related embodiment provides altering the parameter by comparing the ECV to the target volume.

Another exemplary embodiment provided herein is a system for monitoring and regulating radiofrequency (RF) ablation therapy, the system having: an imaging scanner, a RF probe for inserting into a predetermined target volume, a radiofrequency power generator for providing power to the RF probe, a feedback mechanism, and a feedback signal from the scanner, wherein the feedback signal initiates a feedback event.

In a related embodiment of the system provided herein, the imaging scanner is an ultrasound scanner and/or a magnetic resonance scanner. In a related embodiment, the imaging scanner computes at least one of the following: temperature elevation in the target volume, an accumulated thermal dose in the target volume, and an estimated coagulated volume (ECV), to produce a resulting feedback signal from the imaging scanner to the feedback mechanism.

In yet another related embodiment of the system, a comparison of the ECV to the target volume by the feedback mechanism triggers the feedback event. In a related embodiment, the feedback event provides a display of information to the operator for operator approval, and/or an automatic alteration of at least one of the following parameters: RF power, exposure time, and position of the RF probe. In another related embodiment, the display of information to the operator further comprises at least one parameter selected from the group consisting of: an indication of an end of the operation, an indication that reinsertion is required, and generation of an alert if healthy tissue is affected.

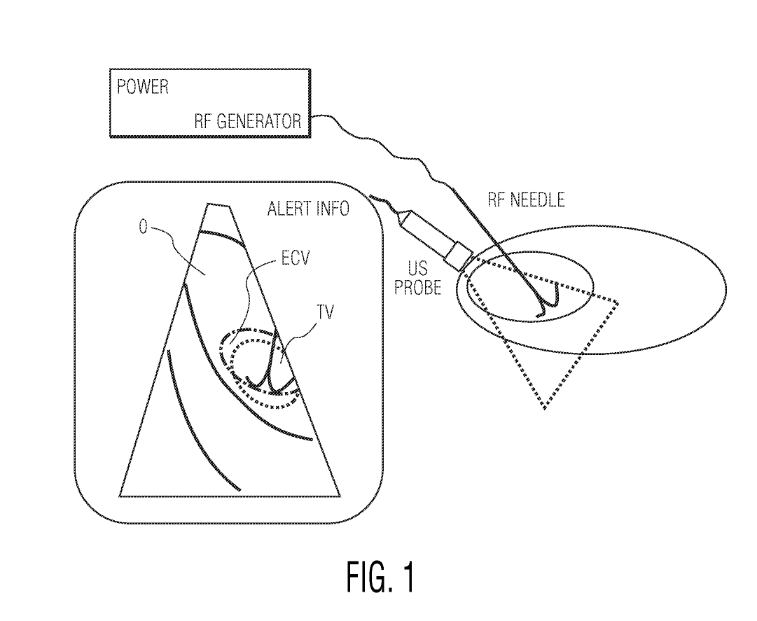

FIG. 1 shows a radiofrequency power generator, a radiofrequency probe penetrating a target volume, an imaging device, consisting of an ultrasound scanner and/or a magnetic resonance scanner or an equivalent imaging device, and an image created by the imaging device of the target volume and surrounding tissue.

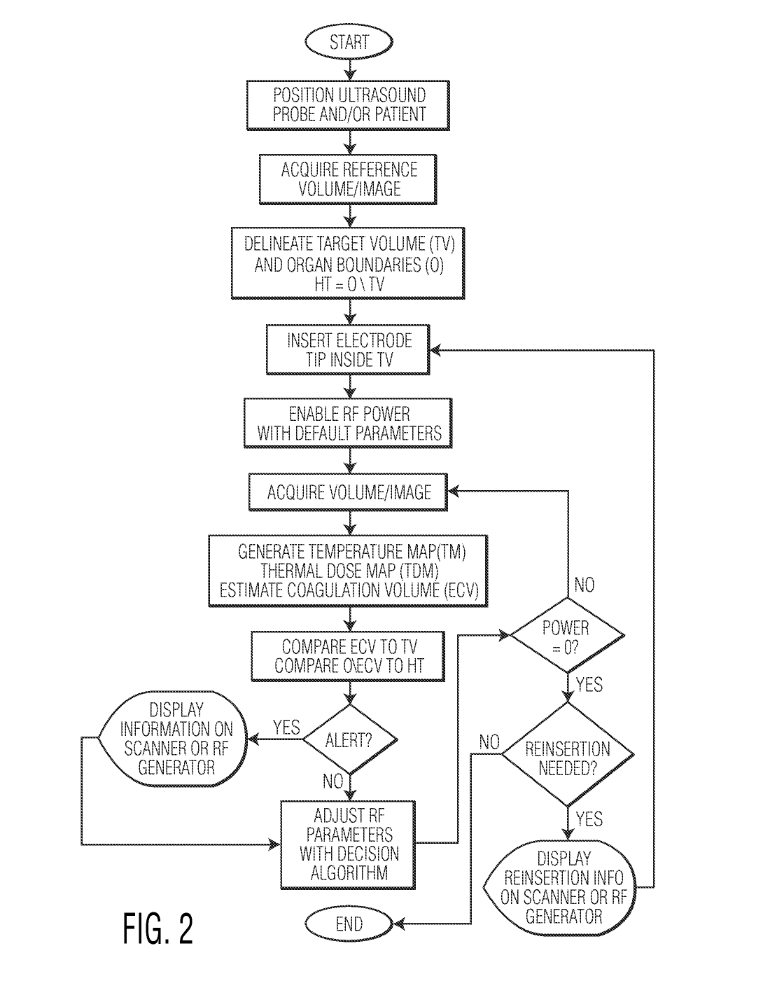

FIG. 2 is a flowchart showing the regulation of a radiofrequency device parameter (power, exposure time, and/or position) using feedback received from an imaging device, consisting of an ultrasound scanner and/or a magnetic resonance scanner or an equivalent imaging device.

The shape and extent of a region of coagulated tissue during or following treatment by RF ablation therapy have been difficult to reproduce using currently available methods. A number of variable biophysical parameters affect the region through alterations in the heat and electrical conduction: a presence of one or more large blood vessels, micro-vessel perfusion or blood volume, prior tissue composition, and formation of excess fibrous tissue in response to a prior history of treatment and/or current treatment. Because of poor reproducibility of the region during current RF procedures, there is a need for methods and devices to visualize the treated region directly, in real time, during the procedure, in order to optimize treatment.

An embodiment of the invention is shown in FIG. 1. An imaging system, e.g. an ultrasound scanner (a portion of which is designated as Ultrasound Probe in FIG. 1), a magnetic resonance scanner, and/or an other imaging device is used to obtain an image of a target volume, for example an organ, a tissue, or a tumor. An RF probe, powered by an RF power generator, is inserted into the target volume. The positioning of the RF probe can be guided using the images obtained by the imaging system. The imaging system also serves as a feedback control mechanism, relaying a feedback signal to the RF power generator, and/or displaying information to an operator.

A first 2-D image or 3-D volume is acquired using an imaging scanner before probe insertion, and the image can further be used to guide the insertion. Boundaries of an organ (designated as O in FIG. 1) and of a target volume to ablate (designated as TV in FIG. 1) are delineated by an automatic and/or a manual method. The organ comprises the target volume and healthy tissue (designated as HT in FIG. 1). Then, a probe is inserted into the target volume. Using default RF input parameters, an RF generator is turned on. The imaging scanner then measures a temperature (based on temperature-dependent ultrasound and/or magnetic resonance parameters), calculates an accumulated thermal dose, and computes an estimated coagulated volume (ECV). The ECV is then compared to the target volume. This comparison is then processed to adjust intensity of the RF current, exposure time and/or spatial position of the RF probe.

The RF probe, which is attached to a power generator, includes a handle and a needle. The handle is held by an operator and the needle is inserted into the target volume. The needle has a distal tip, comprising one or more electrodes. For example, the tip of the needle has a plurality of electrodes, for example three electrodes, and the electrodes curve outward from the tip of the needle into the target volume, branching out from the tip of the needle.

During RF ablation, the probe is inserted into a neoplasm (cancerous tumor) and injects a strong current, which locally heats and destroys tissue. Temperatures above 45-50° C. have been shown to cause denaturation of intracellular proteins and destruction of membranes, producing desired necrosis, or cell death (Haemmerich, D.; Webster, J. G.; Mahvi, D. M.; Engineering in Medicine and Biology Society. Proceedings of the 25th Annual International Conference of the IEEE, 1, pp 134-137, 2003).

Application of RF treatment involves use of a guiding and monitoring imaging modality, such as ultrasound, magnetic resonance, computed tomography, or other equivalent imaging devices. For guidance of initial placement and for adjustment of the RF probe, a method of visualisation of the probe and of the target volume is important. Further, for monitoring and feedback during treatment, it is important that untreated tissue and coagulated volume be clearly distinguishable.

Ultrasound and magnetic resonance imaging techniques both have temperature-dependent properties. For ultrasound, speed of sound waves varies according to temperature. For magnetic resonance, accumulated phase is temperature-dependent. Thus, by manipulating and measuring imaging parameters, temperature within tissue being imaged is determined.

A concept of thermal dose as used herein is a dose parameter that allows comparison of different treatment regimes. Early protocols described thermal treatments in terms of time at a given temperature. However, in the past it has not always been possible to reach a desired or a predetermined temperature level, for reasons related to one or more of the following: technology, patient physiology, and patient comfort. Therefore, a different analytical method, provided herein, is needed.

Based on a time period for which a temperature is held, the estimated thermal dose provides an approximation of an equivalent time at one reference temperature, usually 43° C. To compare a thermal dose accumulated by a tissue subjected to a complex heating regime with a dose it would have experienced had the temperature been held at 43° C., an equivalent time is calculated.

Experimental studies provided herein yield the following model for calculating thermal dose:

D(x,t)=∫β(T(x,t)−T₀)^{(T(x,t)-T}^{₀}^{)/ΔT}dt, wherein:
D(x,t) is the thermal dose, wherein (x is a position, t is a time)
T(x,t) is a spatially and temporally varying tissue temperature,
T₀=43° C., a reference temperature,
ΔT=1 K
β(T(x,t)−T₀)=2 for T(x,t)>T₀>0, and
   β(T(x,t)−T₀)=4 for T(x,t)≦T₀.

From an estimated thermal dose, a calculation of an estimated coagulated volume (ECV) can then be made. In addition to the estimated thermal dose, a known approximate organ-specific value of a thermal dose corresponding to 100% organ necrosis is used (for hepatocytes, the most abundant type of cell in the liver, this value is estimated to be 250-350 minutes). For example, for an organ with a specified volume, if an estimated thermal dose corresponds to one tenth of the thermal dose necessary for complete organ necrosis, the imaging device will compute an ECV corresponding to one tenth of the volume of the organ.

Once an ECV has been calculated, a feedback system compares the ECV to the target volume, and adequacy features are extracted. The adequacy features are user-adjustable to allow for a specified surgical margin of error in order to obtain necrosis of the entire target volume and to have treated a tumor for an adequate thermal dose. Based on those features, decision rules are applied to control an RF power system automatically, or to display information to the operator, including but not limited to: an end of operation, a requirement of reinsertion, and/or a generation of an alert if healthy tissue is affected.

It will furthermore be apparent that other and further forms of the invention, and embodiments other than the specific and exemplary embodiments described above, may be devised without departing from the spirit and scope of the appended claims and their equivalents, and therefore it is intended that the scope of this invention encompasses these equivalents and that the description and claims are intended to be exemplary and should not be construed as further limiting.

1. A method for monitoring and regulating radiofrequency (RF) ablation therapy, the method comprising:
providing an image of a target volume and surrounding tissue using an imaging scanner, and inserting an RF probe into the target volume;
generating an RF current to heat the target volume near at least one tip of the RF probe, and using imaging data as feedback; and
altering at least one parameter selected from the group consisting of: RF power, exposure time, and position of the RF probe, in response to an imaging feedback signal transmitted to an RF power generator and/or displayed to an operator.

2. The method according to claim 1, wherein the scanner comprises an ultrasound scanner and/or a magnetic resonance scanner.

3. The method according to claim 1, wherein the image comprises an ultrasound real-time image and/or a magnetic resonance image.

4. The method according to claim 1, wherein a boundary of the target volume is determined by an automatic method and/or a manual method.

5. The method according to claim 1, wherein the image guides inserting or adjusting placement of the RF probe in the target volume.

6. The method according to claim 1, wherein altering the parameter further comprises computing a temperature elevation in the target volume.

7. The method according to claim 6, wherein the temperature elevation is derived from the image.

8. The method according to claim 7, wherein the temperature elevation is measured through temperature-dependent changes in imaging properties of tissue.

9. The method according to claim 1, wherein altering the parameter further comprises computing an estimated accumulated thermal dose.

10. The method according to claim 9, wherein the estimated accumulated thermal dose is derived from the image.

11. The method according to claim 1, wherein altering the parameter further comprises estimating a location or a volume of a thermally affected region, to calculate an estimated coagulated volume (ECV).

12. The method according to claim 11, wherein the ECV is derived from the image.

13. The method according to claim 1, wherein altering the parameter further comprises comparing the ECV to the target volume.

14. A system for monitoring and regulating radiofrequency (RF) ablation therapy comprising:
an imaging scanner;
a RF probe for inserting into a predetermined target volume;
a radiofrequency power generator for providing power to the RF probe;
a feedback signal from the scanner; and
a feedback mechanism, wherein the feedback signal initiates a feedback event, thereby monitoring and regulating RF ablation therapy.

15. The system according to claim 14, wherein the imaging scanner comprises an ultrasound scanner and/or a magnetic resonance scanner.

16. The system according to claim 14, wherein the imaging scanner computes at least one of the following: temperature elevation in the target volume, an accumulated thermal dose in the target volume, and an estimated coagulated volume (ECV), to produce a resulting feedback signal from the imaging scanner to the feedback mechanism.

17. The system according to either claim 16, wherein a comparison of the ECV to the target volume by the feedback mechanism triggers the feedback event.

18. The system according to claim 17, wherein the feedback event comprises a display of information to the operator for operator approval, and/or an automatic alteration of at least one parameter selected from the group consisting of: RF power, exposure time, and position of the RF probe.

19. The system according to claim 18, wherein the display of information to the operator further comprises at least one parameter selected from the group consisting of: an indication of an end of the operation, an indication that reinsertion is required, and generation of an alert if healthy tissue is affected.
